(19) United States
(12) Patent Application Publication
(10) Pub. No.: US 2023/0013045 A1
(43) Pub. Date: Jan. 19, 2023

(54) Skin Mesher

(71) Applicant: Exsurco Medical, Inc., Wakeman, OH (US)

(72) Inventors: Logan Eggert, Salt Lake City, UT (US); Jared L. Koch, Fox Point, WI (US); Matthew R. Cerreto, Sullivan, OH (US); Amber F. McAfee, Woodstock, GA (US); Taylor L. Warrick, Cleveland, OH (US)

(21) Appl. No.: 17/879,613

(22) Filed: Aug. 2, 2022

Related U.S. Application Data
(60) Provisional application No. 63/211,151, filed on Jun. 16, 2021.

(51) Int. Cl. A61B 17/322

(52) U.S. Cl. CPC A61B 17/322; A61B 2017/3225; A61B 2017/00969

(57) ABSTRACT

A skin mesher apparatus and method including a cover with a crank and a base attached to the cover which encloses a cutting roller and a propulsion roller wherein the cutting roller and propulsion roller are configured vertically align with each other to communicate with the crank and the cutting roller includes a stacked repeating pattern of a first cutting blade with teeth and notches and a second cutting blade with teeth and notches, wherein the second cutting blade is in a mirrored configuration to the first cutting blade.

### CROSS REFERENCE TO RELATED APPLICATIONS

U.S. Provisional application No. 63/211,151, filed on Jun. 16, 2021.

### BACKGROUND OF THE INVENTION

### 1. Field of the Invention

Embodiments of the present disclosure relate generally to the field of skin graft meshers.

### 2. Background

Skin grafts are often prepared by meshing machines during a medical procedure for immediate use on a patient, wherein the meshing machine applies small cuts in a desired configuration to a skin graft so that it may be expanded to a desired state when applied to a target site on a patient. Current meshing machines have various drawbacks which reduce their efficacy for their intended purpose. Some of these include the inability to easily mesh different types of grafts, such allograft, autograft, and skin substitute material using the same machine. Further, current machines suffer from the inability to be taken apart for cleaning and sanitization quickly and effectively, are unable to process grafts wider than 4 inches, and often have complex ineffective procedures for loading and performing a meshing operation on graft material (including slipping or movement of the machine during operation). Additionally, current solutions lack the ability to quickly change graft incision patterns when desired without the need for numerous tools. A solution which solves these noted problems is needed.

### SUMMARY OF THE INVENTION

The invention relates in one embodiment to a skin meshing apparatus including a cover with a crank and a base attached to the cover which encloses a cutting roller and a propulsion roller. The cutting roller and propulsion roller are configured to vertically align with each other and the cutting roller is in communication with the crank and propulsions roller, wherein the crank causes both rollers to rotate. The cutting rolling and propulsion roller are designed to move a carrier tray with skin material or a tissue on the top surface through the pair of rollers, thereby cutting slits in the skin material for application to a patient. Further, the cutting roller is designed to protect the blades from damage if the cutting roller is dropped.

The invention relates in another embodiment to a skin meshing apparatus which includes a cutting rollerwith a stacked repeating pattern of a first cutting blade with teeth and notches and a second cutting blade with teeth and notches, wherein the second cutting blade is in a mirrored configuration to the first cutting blade.

The invention relates in another embodiment, to a method of assembling a skin mesher easily and quickly for cleaning and sanitization, as well for quickly changing graft incision patterns by changing the cutting roller.

### BRIEF DESCRIPTION OF THE DRAWINGS

FIG. 1 is a top perspective view of an embodiment of the present invention;

FIG. 2 is a rear perspective view of an embodiment of the present invention;

FIG. 3 is a bottom perspective view of an embodiment of the present invention;

FIG. 4 is a right perspective view of an embodiment of base 102;

FIG. 4a is a left perspective view of an embodiment of base 102;

FIG. 5 is a top perspective view of an embodiment of propulsion roller 170 and base 102;

FIG. 6 is a top perspective view of an embodiment of the present invention with cover 130 opened to expose cutting roller 160 and propulsion roller 170;

FIG. 7 is a partial exploded view of an embodiment of cutting roller 160;

FIG. 8 is a partial exploded view of an embodiment of left side 162 of cutting roller 160;

FIG. 9 is a front view of an embodiment of first cutting blade 290 and second cutting blade 292 in a 1:1 configuration;

FIG. 10 is a front view of an embodiment of first cutting blade 290 and second cutting blade 292 in a 2:1 configuration;

FIG. 11 is a front view of an embodiment of first cutting blade 290 and second cutting blade 292 in a 4:1 configuration;

FIG. 12 is a magnified top view of a cut pattern on skin material 400 of an embodiment of cutting roller 160 in a 1:1 configuration;

FIG. 13 is a right exploded view of an embodiment of propulsion roller 170;

FIG. 14 is a left exploded view of an embodiment of propulsion roller 170;

FIG. 15 is a perspective view of illustrating how an embodiment of crank 150, cutting roller 160 and propulsion roller 170 are configured to work together to cut skin material; and

FIG. 16 is an exploded view of cover 130 and base 102.

### DETAILED DESCRIPTION OF THE INVENTION

For the following defined terms, these definitions shall be applied, unless a different definition is given in the claims or elsewhere in this specification. All numeric values are herein assumed to be modified by the term “about,” whether or not explicitly indicated. The term “about,” in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary.

As used herein, the terms “skin material” or “tissue” are intended to mean any material used to form a skin graft from human, animal and/or synthetic or partially synthetic material. Examples of such material include but are not limited to split-thickness allograft skin and split-thickness autograft skin.

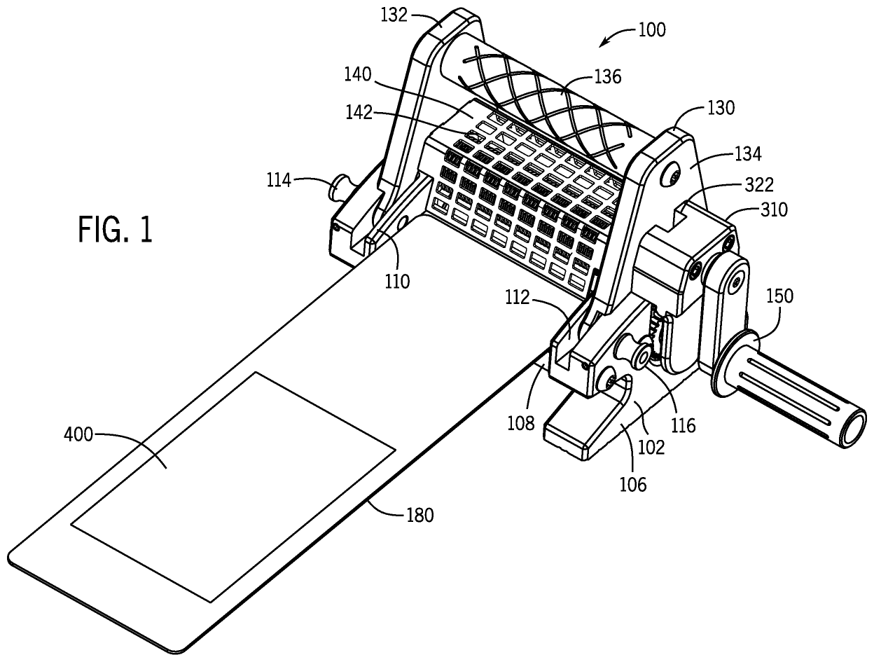

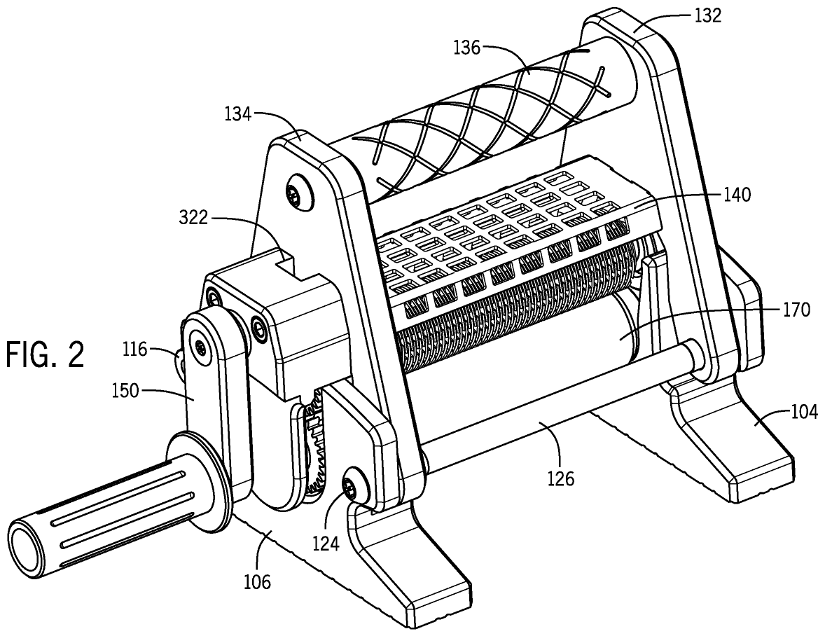

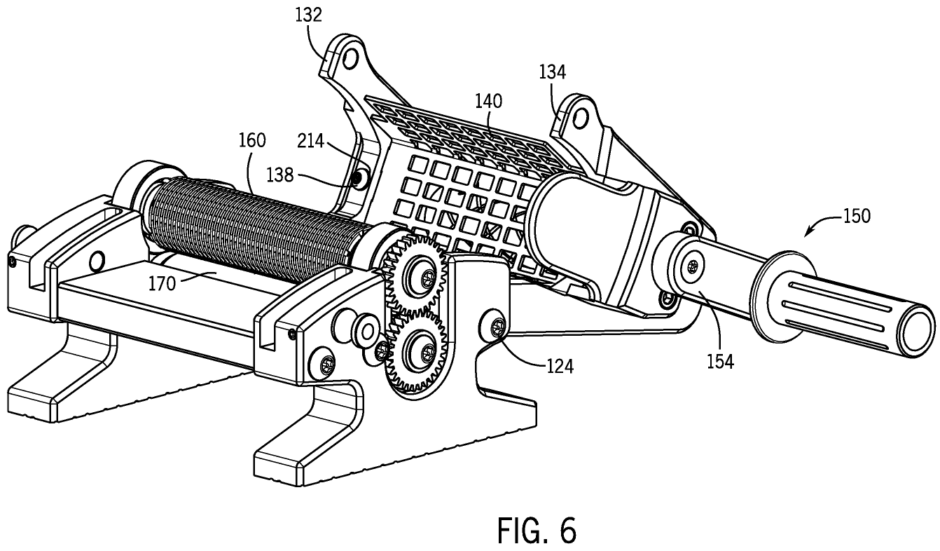

A skin mesher will now be described with references in FIGS. 1-17. Turning to the drawings, where the reference characters indicate corresponding elements throughout the several figures, attention is first directed to FIG. 1 where a top perspective view of an embodiment of the skin mesher is shown, illustrating its composition and the apparatus is generally indicated by reference character 100. Skin mesher 100 is comprised of a base 102, a cover 130, a cutting roller 160 and a propulsion roller 170 (see FIG. 6). Mesher 100 is utilized with a carrier tray 180 configured to hold skin material or a tissue 400 to be cut or perforated by cutting roller 160 wherein carrier tray 180 is propelled through device 100 primarily by propulsion roller 170. Base 102 houses cutting roller 160 and propulsion roller 170 while cover 130 encloses both rollers and is attached to base 102 by at two hinge pin 122 and 124 (see FIG. 2) and additionally houses crank 150. Crank 150 is configured so when it is rotated by a user, cutting roller 160 and propulsion roller 170 also rotate, thereby drawing in carrier tray 180 with skin material or tissue 400 stretched or laid flat on the top surface (which may be smooth or textured if desired), wherein cutting roller 160 acts to cut and perforate the skin material or tissue 400 with slits of a size, shape and configuration determined by cutting blades (290 and 292 described below) located within cutting roller 160. In addition, base 102 further comprises a left leg 104 and a right leg 106 located on either side of base 102 and an infeed tray 108 configured to receive carrier tray 180 and located between left leg 104 and right leg 106 of the front of base 102 and extends away from base 102 at a height which allows carrier tray 180 to slide across infeed tray 108 and in between cutting roller 160 and propulsion roller 170. Infeed tray 108 further comprises a left guide rail 110 and a right guide rail 112 located on opposing sides of infeed tray 108 and configured to further guide carrier tray 180. The ends of guide rails 110 and 112 may be mitered or rounded to allow carrier tray 180 to more easily slide in between guide rails 110 and 112. Further, infeed tray 108 may be beveled or rounded in order to also assist in the feeding of carrier tray 180 onto infeed tray 108. Infeed tray 108 may be any desired height but in the present embodiment is about 1.8 inches from the bottom of base 102. Infeed tray 108 is secured to left leg 104 and right leg 106 by at least one fastener 456 (preferably 2) extending into the portion of infeed tray 108 in communication with left leg 104 and at least one fastener 456 (preferably 2) extending into the portion of infeed tray in communication with right leg 106. Base 102 is ideally made of 6061 aluminum but may be made from any material that is easy to sterilize and is clean room or operating room compatible. Carrier tray 180 is ideally made of plastic and can be created in any length as well as cut by a user with access to some scissors but is generally rectangular in shape and equal to or smaller than the width of infeed tray 108, which is generally 4 inches or greater. Carrier tray 180 is configured so that either the top or bottom surface can be positioned face up for skin material 400 to adhere to for cutting, making carrier tray 180 compatible with mesher 100 regardless of whether the top or bottom surface of carrier tray 180 is used for skin material 400.

Base 102 further comprises left lock pin 114 located on the outside wall of left leg 104 and right lock pin 116 located on the outside wall of right leg 106. Lock pin 114 extends into left leg 104 and at least a portion of cover 130 and lock pin 116 extends into right leg 106 and at least a portion of cover 130, thereby locking cover 130 to base 102 for operation of mesher 100. Preferably lock pins 114 and 116 are each secured to their respective leg by a spring-loaded ball assembly pushing against the shaft of the lock pin (and located within each leg), wherein the shaft of each lock pin has a slot milled in it where each respective ball assembly extends into, thereby defining the range of movement of each lock pin 114 and 116. When Lock pins 114 and 116 are pulled away from base 102 each pin stays disengaged from cover 130 until a user manually pushes them back to reengage a closed cover 130, thereby reducing potential operator confusion and error during generally stress laden operative procedures. To open mesher 100 left lock pin 114 can be pulled out/away from left leg 104 while right lock pin 116 is also pulled out/away from right leg 106 while cover 130 is lifted away from base 102. Opening cover 130 (see FIG. 6) exposes cutting roller 160 and propulsion roller 170 for service or installing another cutting roller or propulsion roller.

Cover 130 comprises a left support 132, a right support 134, a handle 136 and a crank 150, wherein the top inner surface of left support 132 is in contact with one end of handle 136 and the top inner surface of right support 134 is in contact with the other end of handle 136. Handle 136 allows a user to pick up mesher 100 and move it easily and it acts as a support for a user while they turn crank 150 to operate cutting roller 160 on a skin material or tissue 400 located on carrier tray 180. Handle 160 may be textured and in a shape desirable to make mesher 100 easy to pick up and hold. Cover 130 further comprises a guard 140 located below and in parallel alignment with handle 136 and secured to the lower inner surface of left support 132 on one side of guard 140 and secured to the lower inner surface of right support 134 on a second side of guard 140. Guard 140 is generally “J” shaped with rectangular sections and bent to circumscribe the top half of cutting roller 160 and extends from about the middle of left guide rail 110 and right guide rail 112 to cover the top of cutting roller 160 while also providing enough space for a user to access and remove any tissue material 400 which may stick to cutting roller 160 blades during cutting. Guard 140 further comprises a plurality of openings 142 formed therethrough and patterned along the entire surface of guard 140 which are small enough to prevent a user's fingers from touching or getting caught in-between cutting roller 160 and guard 140 or propulsion roller 170, yet large enough to allow a user to view roller 160 in operation to ensure that skin material 400 being cut does not stick to cutting roller 160, potentially damaging the skin material or tissue 400 (and removing it from carrier tray 180). In the preferred embodiment opening 142 is about 0.375 inches by 0.25 inches in size, but any shape and size may be used which serve the purposes states above. Guard 140 is ideally made of 300 series stainless steel but may be made from any material that is easy to sterilize and is clean room or operating room compatible.

Turning to FIG. 2, a rear perspective view of an embodiment of the mesher 100 is shown. Cover 130 is attached to base 102 by left hinge pin 122 and right hinge pin 124. Left hinge pin 122 is located on the rear side of left leg 104 and right hinge pin 124 is located on the rear side of right leg 106. Left hinge pin 122 extends through left support 132 of cover 130 and screws into one end of cross brace 126 to create a hinge for cover 130 to open and close, thereby allowing cutting roller 160 and propulsion roller to be removed for cleaning or to replace cutting roller 160 with another roller with a different cutting configuration. Likewise, right hinge pin 124 extends through right support 134 and secures into the other side of cross brace 126 to provide another hinge for cover 130 to open and close in relation to base 102. Cross brace 126 keeps the rear of left leg 104 and the rear of right leg 106 in alignment and may additionally act as a guidance point to guide carrier tray 180 when exiting cutting roller 160 and propulsion roller 170. Further, cross brace 126 is ideally cylindrical but could be other shapes as well.

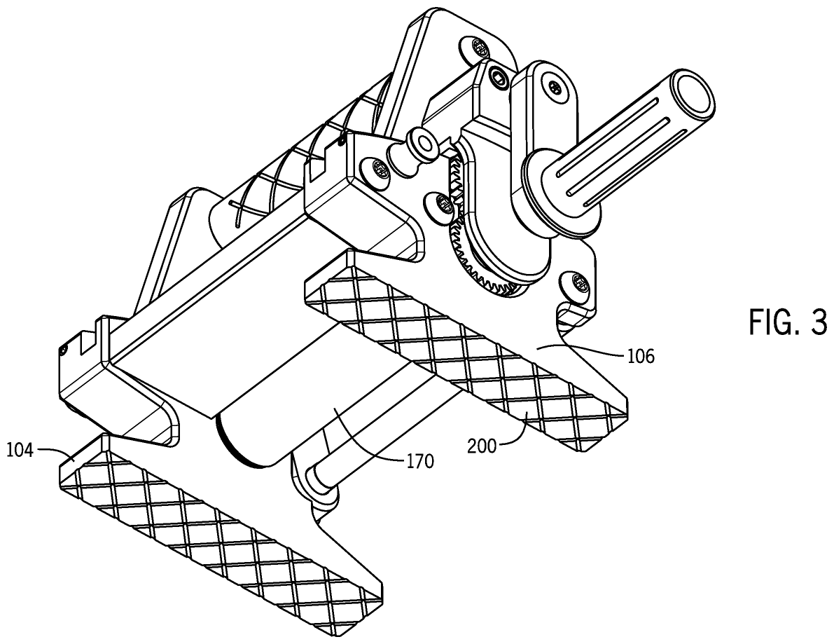

Turning to FIG. 3, a bottom perspective view of mesher 100 is shown. Base 102, left leg 104 and right leg 106 may include a texture 200 on their bottom surfaces, preferably formed integral with the bottom surfaces, wherein texture 200 is configured to provide additional traction to keep mesher 100 stationary while in operation (which includes force applied to handle 136 as well as applied to crank 150). In the current embodiment texture 200 includes a knurled cross-hatched or diamond pattern but any desired pattern could be utilized which improves base 102 adherence to a flat surface in order to reduce movement during use.

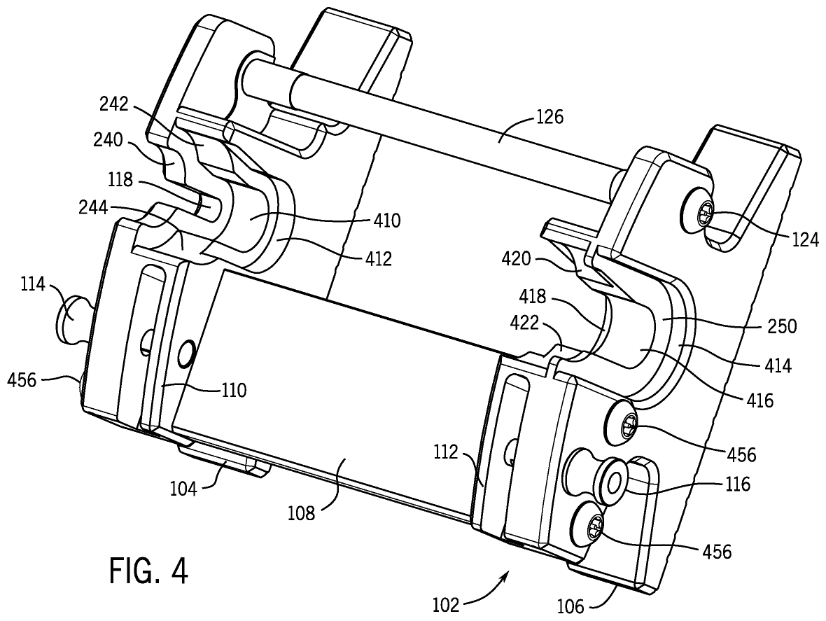

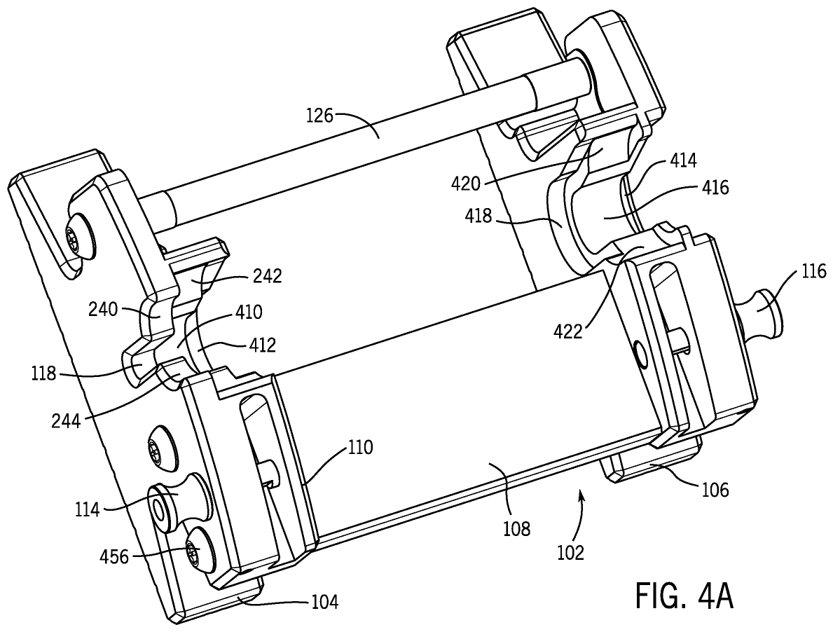

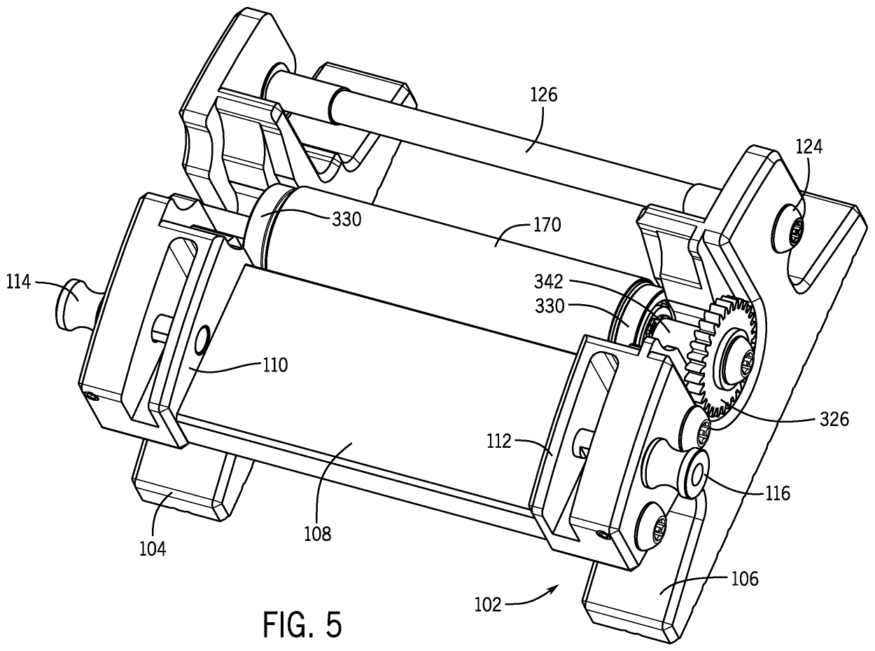

Turning first to FIG. 4 and FIG. 4a, a right and left perspective view of an embodiment of base 102, and then to FIG. 5, a top perspective view and a top view of propulsion roller 170 and base 102 is shown. The left leg 104 of base 102 further comprises a left cradle 240 and the right leg 106 further comprises right cradle 250 which are configured to seat propulsion roller 170 as well as cutting roller 160 (not shown), wherein cutting roller 160 is seated in vertically alignment with, and located above, propulsion roller 170. Left cradle 240 comprises a first slot 118 formed in the outside surface of left leg 104 and configured to hold an axle rod 338 of propulsion roller 170 (described below), a second slot 410 located next to first slot 118 and configured to hold a bearing 266 of cutting roller 160 (described below) via a first circular arc 242 and a second circular arc 244, and a third slot 412 located next to second slot 410 and formed in the inside surface of left leg 104 and configured to hold a bearing 330 of propulsion roller 170 (described below). First slot 118, second slot 410, and third slot 412 are currently each configured in a different curvilinear “U” shape, however second slot 410 is deeper than first slot 118 and third slot 412 is deeper than second slot 410, but other shapes may be utilized to achieve the same purpose. First circular arc 242 and a second circular arc 244 are located toward the top of second slot 410 and configured to hold a bearing 266 of cutting roller 160 at a height where the cutting roller 160 can perform a cutting operation on a tissue sample 400 moving through mesher 100 on a carrier tray 180.

Right cradle 250 further comprises an outer slot 414 adjacent to the outside surface of right leg 106 and configured to allow propulsion roller gear 326 (see FIG. 5 and described below) and at least a portion of first gear 272 of cutting roller 160 (described below) to rotate freely, a middle slot 416 next to outer slot 414 and configured allow spacer 342 of propulsion roller 170 to extend through it and to also hold a bearing 266 of cutting roller 160 (see FIG. 5 and described below) via a third circular arc 420 and a fourth circular arc 422, and an inner slot 418 located adjacent the inside surface of right leg 106 and configured to hold a bearing 330 of propulsion roller 170 (described below). Inner slot 418 and third slot 412 are configured to each hold a bearing 330 of propulsion roller 170 so propulsion roller 170 is held horizontal and parallel with any surface base 102 is placed upon. Third circular arc 420 and a fourth circular arc 422 are located toward the top of middle slot 416 and configured to hold a bearing 266 of cutting roller 160 at a height where the cutting roller 160 is held parallel to propulsion roller 170 and can perform a cutting operation on a tissue sample 400 moving through mesher 100 on carrier tray 180 and allowing propulsion roller 170 and cutting roller 160 to be secured vertically in line with one another by cover 130 (see FIG. 6).

Outer slot 414, middle slot 416, and inner slot 418 are each currently configured in a curvilinear “U” shape, however outer slot 414 and inner slot 418 are preferably the same depth and deeper than middle slot 416, however other shapes may be utilized to achieve the same purpose. In the current embodiment, bearings 266 of cutting roller 160 are larger in diameter than bearings 330 of propulsion roller 170 and bearings 266 and 330 do not touch when cutting roller 160 and propulsion roller 170 are seated in left cradle 240 and right cradle 250. This prevents a user from installing cutting roller 160 into the designated position of propulsion roller 170 and vice-versa. It is contemplated, if desired, that bearings 266 and 330 could touch in an alternative embodiment.

Turning to FIG. 6, a top perspective view of mesher 100 with cover 130 opened, exposing cutting roller 160 and propulsion roller 170 is shown. Propulsion roller 170 is positioned in base 102 by left cradle 240 formed in left leg 104 and right cradle 250 formed in right leg 106 (see FIG. 4). Propulsion roller 170 is easily removable from left cradle 240 and right cradle 250 by a user's fingers, allowing not only cutting roller 160 to be changed but also propulsion roller 170 without the need for any tools. Cover 130 further comprises at least one height positioning stud 138, but preferably two. One height positioning stud 138 is located on the interior surface of left leg 132 and a second height positioning stud 138 may be located on the interior surface of right leg 134. Each stud 138 is positioned near both ends of cutting roller 160, thereby sandwiching bearing 266 (described below) of cutting roller 160 between each stud 138 and left cradle 240 or right cradle 250 while stud 138 is currently cylindrical, other shapes could be utilized. Height positioning stud 138 is preferably a tamper resistant spanner screw 210 and may further comprise one or more shims 214. If desired, stud 180 may be adjusted by the manufacturer to create a specific distance between propulsion roller 170 and cutting roller 160, such as to accommodate a specific skin material or tissue 400 thickness. In addition, due to manufacturing tolerance ranges of mesher 100 components, adjustment of one or more studs 138 may be required to ensure a very tight fit/compression of each stud 138 on each corresponding bearing 266 of cutting roller 160. Each shim 214 is preferably 0.002 inches in height but may be any height desired. Height positioning stud 138 fastens to cover 130 to secure one or more shims 214 between stud 138 and the inner surface of cover 130.

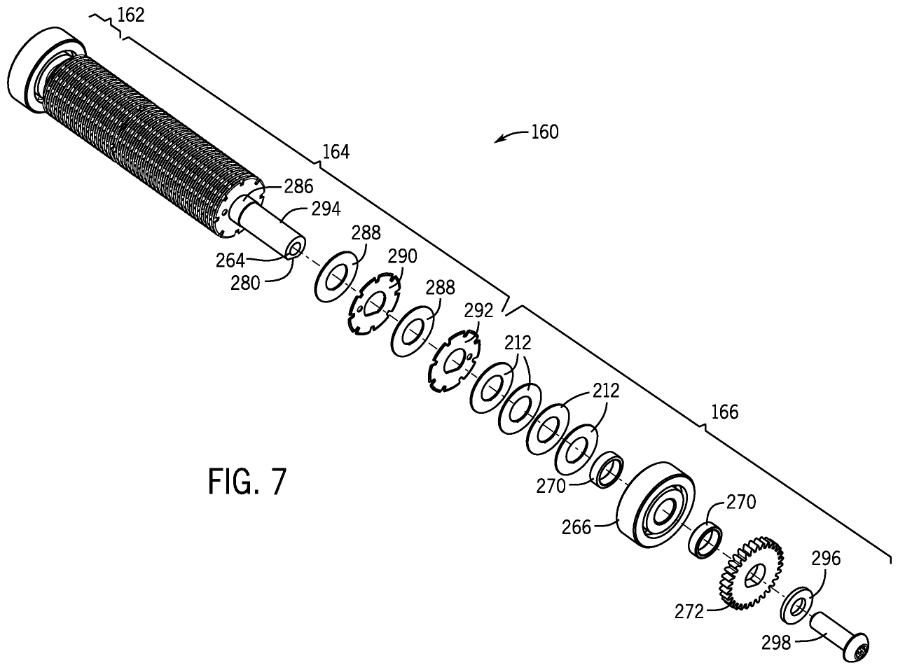

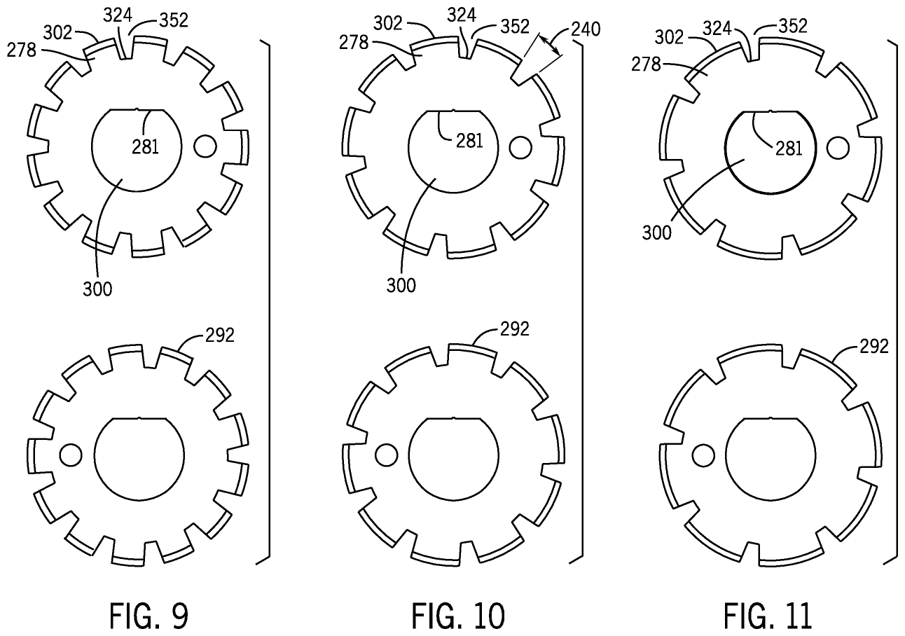

Turning to FIG. 7, a partial exploded view of an embodiment of cutting roller 160 is shown. Cutting roller comprises a left side 162, a center portion 164, and a right side 166. Center portion 164 is comprised of shaft 286 which has two ends 294 which are mirror images of each other and a series of spacers 288 and cutting blades 290 and 292. End 294 has a diameter that is the same as or slightly smaller than the rest of shaft 286 and is configured to slidably communicate with each bearing 266. End 294 further comprises an opening formed in the top and configured to secure a fastener 298 (a tamper resistant spanner screw in the current embodiment). Shaft 286 is generally cylindrical in shape with a key or flat side 280. Key 280 allows cutting blades to slide and secure onto shaft 286 in one of two possible positions, regular or flipped (see FIGS. 9-11). Center portion 164 is comprised of a repeating pattern or series of at least a first blade 290, a spacer 288 (or multiple spacers), and second blade 292; all slid onto shaft 286, followed by another spacer 288 and another series of first blade 290, spacer 288, and second blade 292, preferably repeating the spacer and series pattern along the entire length of the shaft to create a blade stack. Shaft 286 is ideally made of 400 series stainless steel but may be made from any material that is easy to sterilize and is clean room or operating room compatible.

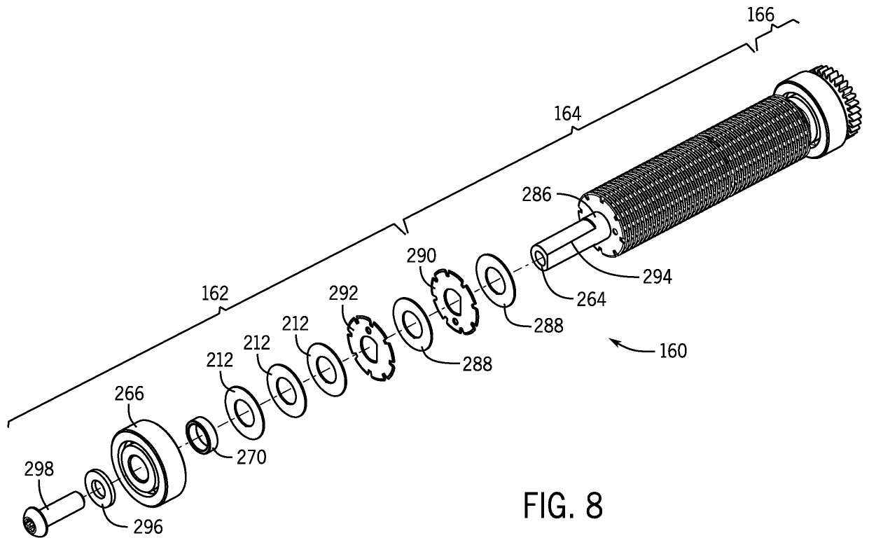

Right side 166 comprises at least one shim 212 (in the current embodiment a washer) slid onto end 294, followed by a spacer 270, bearing 266, another spacer 270, a first gear 272, a washer 296, and a fastener 298, wherein fastener 298 is configured to secure within aperture 264 in end 294. The quantity of shims 212 used (4 in the current embodiment) will vary according to the manufacturing tolerances of each cutting blade 290 and 292 used in the blade stack. Bearing 266 is larger in diameter (preferably about 1.5 inches) than the diameter of cutting blades 290 and 292 (preferably about 1.2 inches) and first gear 272 in order to prevent potential damage to blades 290 and 292 if cutting roller 160 is dropped and/or cutting blades 290 and 292 collide with a surface, such as a floor or wall. Once the end of shaft 286 has been reached with the repeating pattern or series of spacers 288, cutting blades 290/292, bearing 266, spacers 270, first gear 272, and washer 296, they are all compressed onto shaft 286 when fastener 298 is secured to shaft 286. Turning to FIG. 8, a partial exploded view of left side 162 of cutting roller 160 is shown. Left side 162 comprises at least one shim 212 (in the current embodiment a washer), a spacer 270, a bearing 266, and washer 296 slid onto end 294 and secured onto end 294 by a tamper resistant fastener 298, where fastener 298 configured to secure within aperture 264. The quantity of shims 212 used (4 in the current embodiment) will vary according to the manufacturing tolerances of each cutting blade 290 and 292 used in the blade stack.

Turning to FIG. 9, a front view of an embodiment of first cutting blade 290 and second cutting blade 292 with a 1:1 cutting, or expansion ratio is shown. Mesher 100 can change between cutting ratios quickly and easily simply by changing out cutting roller 160 with another roller with a different blade configuration. Multiple cutting or expansion ratio options let users, such as surgeons, tailor a skin material graft to each specific patient and wound site during a procedure. First cutting blade 290 comprises a plurality of teeth 278 which circumscribe blade 290. Each tooth 278 has a cutting edge 302 configured to cut or perforate skin material 400. Further, between each tooth 278 is a notch 352 with a notch base 324. The relationship between the length of cutting edge 302 and the length of notch base 324 allows for various first cutting blade 290 and second cutting blade 292 embodiments which may be desirable to a user for a specific application of skin material 400. For example, in a 1:1 radio of cutting edge 302 length to notch base 324 length, the preferred cutting edge 302 length is about 0.1 inches and notch base 324 length is about 0.1 inches. The angle between the blade teeth 278 of first cutting blade 290 and the corresponding blade teeth 278 of second cutting blade 290 (when slide onto shaft 286) is preferably about 10.6 degrees. For a 2:1 ratio the preferred cutting edge 302 length is about 0.2 inches and notch base 324 length is 0.1 inches (see FIG. 10). The angle between the blade teeth 278 of first cutting blade 290 and the corresponding blade teeth 278 of second cutting blade 290 (when slide onto shaft 286) is preferably 15 degrees. For a 4:1 ratio the preferred cutting edge 302 length is about 0.4 inches and notch base 324 length is about 0.1 inches (see FIG. 11). The angle between the blade teeth 278 of first cutting blade 290 and the corresponding blade teeth 278 of second cutting blade 290 (when slide onto shaft 286) is preferably about 22.5 degrees. Likewise, the spacing between each cutting blade may be altered by adding additional spacers 288 between each first cutting blade 290 and second cutting blade 292 onto shaft 286.

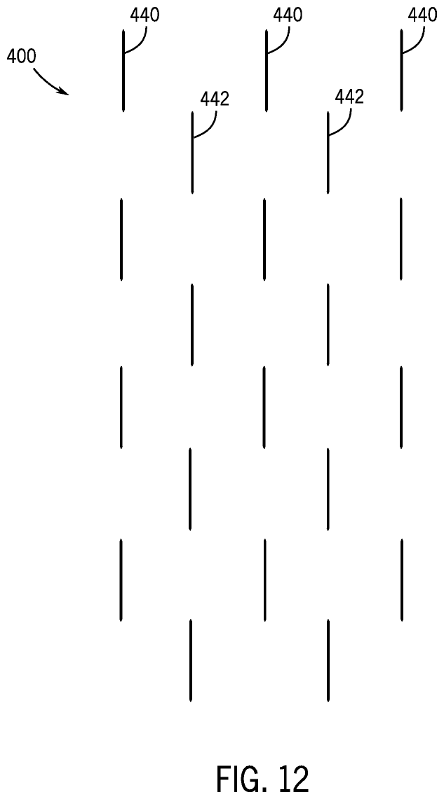

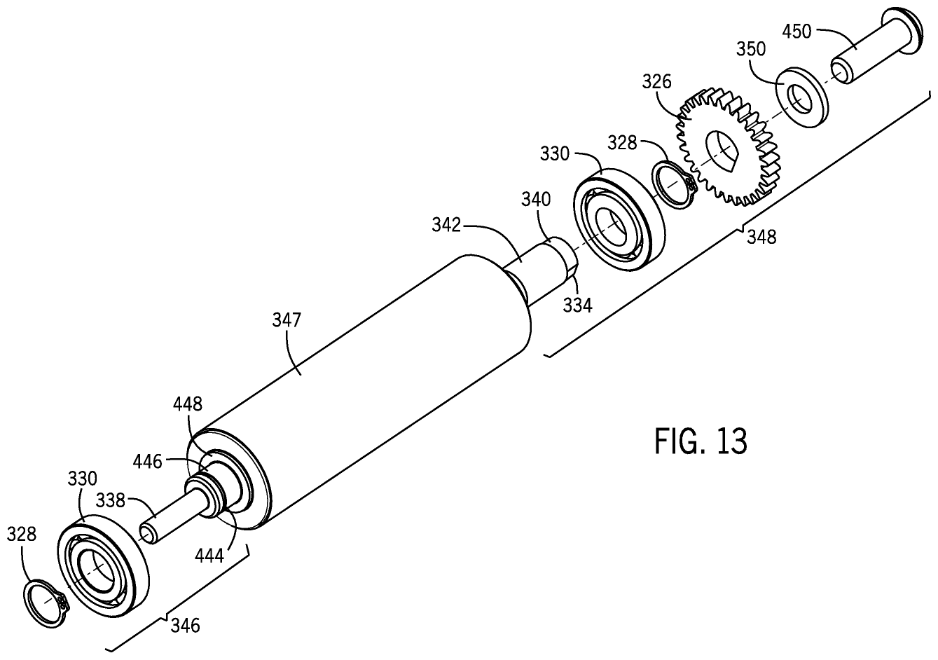

In the preferred embodiment each first cutting blade 290 and second cutting blade 292 has an outer diameter of 1.2 inches with each cutting tooth 278 (and notch 352) a height of about 0.23 inches, while the sides of notch 352 (which are also the sides of tooth 278) create a 24 degree angle (noted on FIG. 10). This angle helps to prevent skin material 400 from sticking to cutting edge 302 of each tooth 278. In addition, in a specific embodiment, second cutting blade 292 has the same shape and configuration as first cutting blade 290 however the orientation of teeth 278 are offset to first cutting blade 290, which allows for a staggered cut pattern on skin material 400 as shown in FIG. 12 where first cut 440 from cutting blade 290 and second cut 442 from second cutting blade 292 is shown. In some cases, such as in the 1:1 ratio, first cutting blade 290 may by identical to second cutting blade 292, except that second cutting blade 292 is flipped in its orientation before it is slid onto shaft 286. First cutting blade 290 and second cutting blade 292 also include an aperture 300 formed therethrough the same shape as shaft 286, which includes a flat portion 281 to match key 280 on shaft 286. The shape of shaft 286 and aperture 300 ensures blade 290 can slide onto shaft 286 in only one of two possible configurations, regular or flipped. Cutting blades 290 and 292 are ideally made of 400 series stainless steel but may be made from any material that is easy to sterilize and is clean room or operating room compatible. Further, cutting blades 290 and 292 may be permanently marked, color coded, and/or marked (such as by a shape punched through the blade, such as a circle in the current embodiment) to convey their proper orientation on cutting roller, cutting ratio, and/or blade number identification (either first cutting blade 290 or second cutting blade 292) to ensure proper assembly of cutting roller 160.

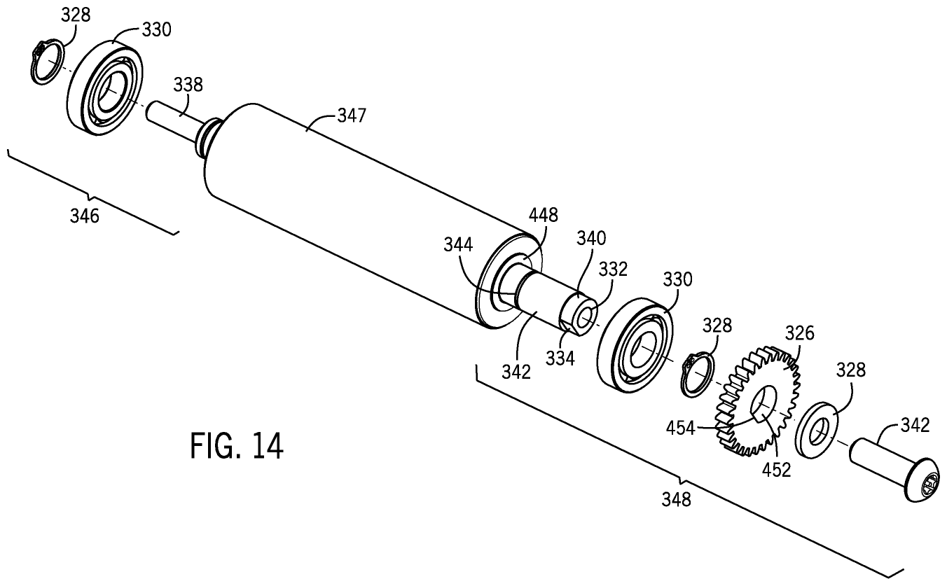

Turning to FIG. 13 and FIG. 14, a left exploded view of propulsion roller 170 and a right exploded view of propulsion roller 170 is shown. Propulsion roller 170 comprises a left side 346, center 347 and right side 348. Center 347 is preferably a solid piece of material, cylindrical in shape and ideally equivalent in outer diameter as cutting blades 290 and 292 on cutting roller 160 but does not have to be. Center 347 comprises a cross-hatched or diamond texture that is knurled to ensure carrier tray 180 is advanced through mesher 100 by propulsion roller 170.

Left side 346 comprises an axle rod 338 extending from the center of the left end of center 347. Left side 346 further comprises a first portion 446 extending from the center of the left end of center 347 with a larger diameter than axle rod 338 and configured to hold bearing 330 so that bearing 330 rotates around first portion 446. First portion 446 further comprises a groove 444 circumscribing first portion 446 and configured to receive a retainer clip 328 in order to retain bearing 330 in communication with first portion 446. Left side 346 further comprises a second portion 448 larger in diameter than first portion 446 and extending from the center of the left end center of roller 170 and configured to provide distance between bearing 330 and center 347 to provide space for cleaning fluid when cleaning roller 170 after use. While axle rod 338, first portion 446, and second portion 448 are formed integral with center 347, they may be separate if desired.

Right side 348 comprises a shaft 342 extending from the center of the right end of center 347 and configured to hold bearing 330 so that bearing 330 rotates around shaft 342. Shaft 342 further comprises a grove 344 circumscribing shaft 342 and configured to receive a retainer clip 328 in order to retain bearing 330 in communication with shaft 342. Shaft 342 further comprises end section 340 which may be the same or smaller diameter than the rest of shaft 342 and further comprises flat section 334 and aperture 332 formed on the top thereof and extending into shaft 342. Right side 348 further comprises gear 326 which includes aperture 452 with a flat section 454, wherein aperture 452 and flat section 454 are configured to be in communication with end section 340 and flat section 334 so that gear 326 can be secured to end section 340 by fastener 450 which is configured to secure into aperture 332, thereby allowing gear 326 to rotate center 347. Gear 326 is configured to be in rotational communication with first gear 272 of cutting roller 160. In addition, right section 348 further comprises spacer 328 which fits between gear 326 and fastener 450.

When assembled, gear 326 of propulsion roller 170 is seated in outer slot 414 of base 102 (see FIGS. 4-5) so a user can visually see if gear 326 is aligned with first gear 272 of cutting roller 160, to make it easier for a user to install and remove both cutting roller 160 and propulsion roller 170 as well as to make cleaning/sterilization of base 102 more efficient. Center 347 is ideally made of 300 series stainless steel but may be made from any material that is easy to sterilize and is clean room or operating room compatible. Propulsion roller 170 not only helps feed and secure carrier tray 180 so cutting roller 160 can perforate skin material 400, but it also provides a counter force to cutting roller 160, further ensuring edges 302 of cutting blades 290 and 292 fully puncture skin material 400, which is important for proper healing of skin material 400 when applied to the desired patient site. In addition, propulsion roller gear 326 is configured to operate with first gear 272 of cutting roller 160 which allows cutting rollers with different cutting ratios to be changed out during use and are all compatible with the same propulsion roller 170.

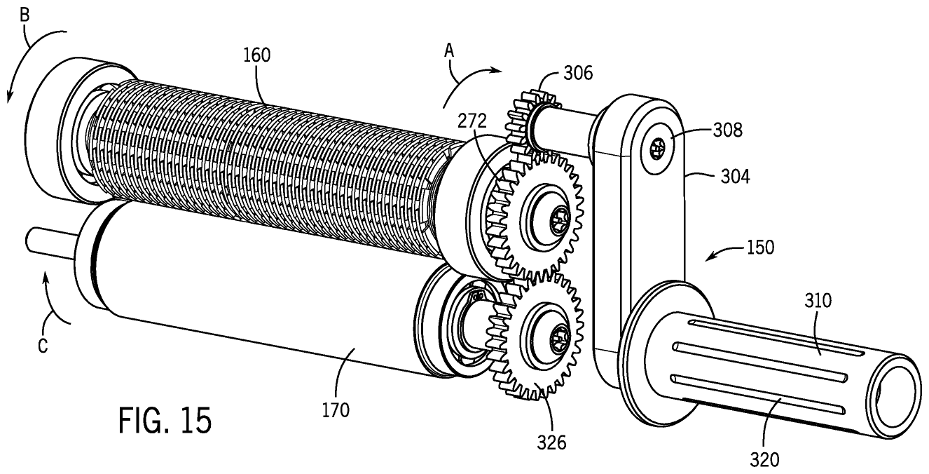

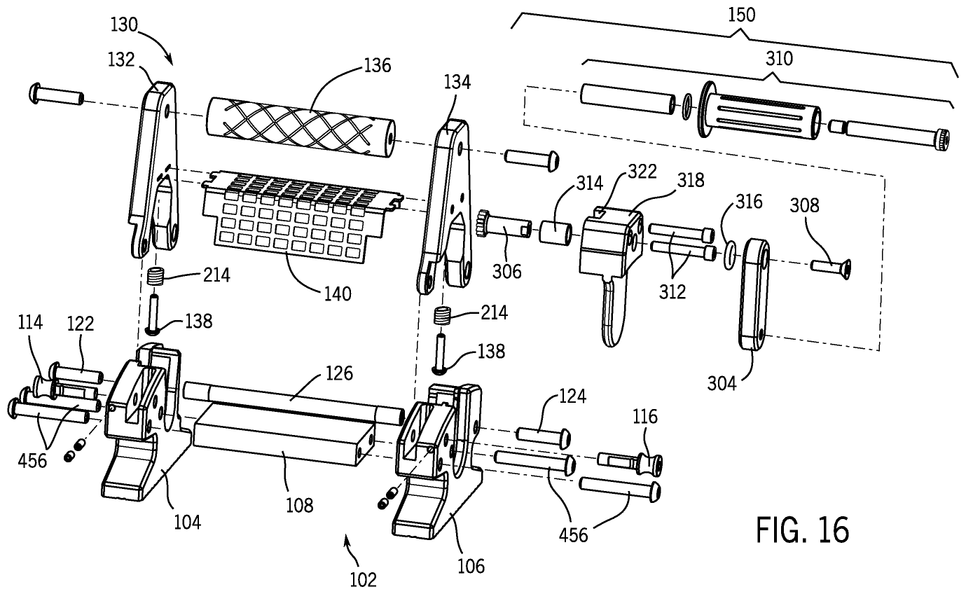

Turning to FIG. 15, a perspective view of how crank 150, cutting roller 160 and propulsion roller 170 are configured to work together to cut skin material 400 is shown. Crank 150 comprises a crank handle 310 attached to a handle arm 304, which is further attached to a drive gear 306 by tamper resistant fastener 308. Crank handle 310 may further comprise a texture 320 which makes it easier for a user to grasp and hold in order to operate crank 150 and drive cutting roller 160 to cut skin material 400. Handle arm 304 may be any desired length and the center of which is distanced from the bottom of base 102 enough to prevent a user from hitting their hand on a table or surface where mesher 100 is located but also spaced enough to provide sufficient torque to cause cutting roller 160 to cut skin material 400. In the present embodiment this is about 3.5 inches. Drive gear 306 is vertically aligned with first gear 272 of cutting roller 160 and gear 326 of propulsion roller 170 wherein drive gear 306 is located on top of cutting roller 160 and configured to be in rotational communication with first gear 272 of cutting roller 160 and first gear 272 is configured to be in rotational communication with gear 326 of propulsion roller 170. Using the arrows indicated you will see when handle 150 is rotated in a clockwise direction A, drive gear 306 drives cutting roller 160 to rotate in a counter-clockwise rotation B, which drives propulsion roller 170 to rotate in a clockwise rotation C, thereby pulling carrier tray 180 with skin material 400 on its top surface in-between cutting roller 160 and propulsion roller 170, and allowing the plurality of first cutting blade 290 and second cutting blade 292 to cut or perforate the skin material 400 in a smooth and continuous manner, as long as crank handle 150 is rotated, until carrier tray 180 and its skin material 400 have proceeded entirely through cutting roller 160 and propulsion roller 170 and out of mesher 100. This configuration allows for precise and consistent cut patterns throughout the entire length of skin material 400. While drive gear 306 is smaller in diameter (presently about 0.7 in) than first gear 272 and gear 326, it can change if different crank drive characteristics are desired (the same applies to the size of first gear 272 and gear 326, which are presently the same at about 1.32 inches). Turning to FIG. 16, an exploded view of cover 130 and base 102 is shown. Crank 150 further comprises a crank housing 318 which is fastened to right leg 134 by at least one screw 312, but preferably 2 (or could be formed integral with right leg 134). Handle 310 is connected to crank arm 304 by a fastener, while crank arm 304 is fastened to drive gear 306 by a fastener 308 (such as a screw), which also sandwiches an 0-ring 316 and a dry sleeve bearing 314, wherein both O-ring 316 and dry sleeve bearing slide over a shaft connected to drive gear 306. O-ring 316 acts as a washer, preventing drive gear 306 and dry sleeve bearing 314 from being able to slide in relation to crank housing 318 and also prevents drive gear 306, or debris from said gear, from grinding on inside of the crank housing 318. Further, crank housing 318 may contain a fluid egress 322 (see FIG. 2 also) to allow fluid flow from unit during cleaning or sterilization. Crank arm 304 in the present embodiment is about 3 inches long.

A method of assembling mesher 100 including opening pulling lock pin 114 and lock pin 116 away from base 102 while lifting cover 130, releasing lock pins 114 and 116, placing propulsion roller 170 in base 102 by aligning bearing 330 of left side 346 within third slot 412 of left cradle 240 of left leg 104 and aligning bearing 330 of right side 348 within inner slot 418 of right cradle 250, placing cutting roller 160 on top of propulsion roller 170 by aligning bearing 266 of left side 162 with first circular arc 242 and second circular arc 244 of left cradle 240 and aligning bearing 266 of right side 166 with third circular arc 420 and fourth circular arc 422 of right cradle 150 and aligning first gear 272 with gear 326, pulling lock pin 114 and lock pin 116 away from base 102 while closing cover 130, and releasing lock pins 114 and 116 to reengage with cover 130.

While the present invention has been described above in terms of specific embodiments, it is to be understood that the invention is not limited to these disclosed embodiments. Many modifications and other embodiments of the invention will come to mind of those skilled in the art to which this invention pertain, and which are intended to be and are covered by both this disclosure and the appended claims. It is indeed intended that the scope of the invention should be determined by proper interpretation and construction of the appended claims and their legal equivalents, as understood by those of skill in the art relying upon the disclosure in this specification and the attached drawings. Likewise, where this document refers to technologies that would be apparent or known to one of ordinary skill in the art, such technologies encompass those apparent or known to the skilled artisan now or at any time in the future.

1. A skin mesher comprising:
a cover comprising a crank;
a base attached to the cover;
a cutting roller; and
a propulsion roller;
wherein the cutting roller and propulsion roller are removable from the base and configured vertically align with one another and are located inside the base and cover and the crank is configured to rotate the cutting roller and the propulsion roller.

2. The mesher of claim 1 wherein the mesher further comprises a carrier tray with a top and bottom surface wherein both the top and the bottom surface are suitable to hold a skin material.

3. The mesher of claim 1 wherein the cutting roller further comprises a shaft and a repeating pattern of at least one first cutting blade, at least one spacer, and at least one second cutting blade slid onto the shaft.

4. The mesher of claim 3 wherein the cutting roller further comprises at least two bearings wherein the bearings have a larger diameter than the first cutting blade and second cutting blade.

5. The mesher of claim 1 wherein the cover further comprises a guard configured around at least a portion of the cutting roller.

6. The mesher of claim 5 wherein the guard is contains a plurality of openings formed therethrough.

7. The mesher of claim 1 wherein the base further comprises a texture on its bottom configured to resist movement.

8. The mesher of claim 1 wherein the base further comprises a removable infeed tray.

9. A skin mesher comprising:
a cover comprising a crank;
a base attached to the cover;
a cutting roller; and
a propulsion roller;
wherein the cutting roller comprises a first cutting blade and a second cutting blade, wherein the first cutting blade and second cutting blade are in a stacked configuration on a shaft of the cutting roller and each blade further comprises at least one notch and at least one tooth.

10. The skin mesher of claim 9 wherein the first cutting blade and second cutting blade further comprise an aperture formed therethrough configured to match the shape of the shaft.

11. The skin mesher of claim 9 wherein the first cutting blade and the second cutting blade are the same shape and the second cutting blade is in a flipped configuration as compared to the first cutting blade on the shaft of the cutting roller.

12. The skin mesher of claim 9 wherein the at least one tooth of the first cutting blade and the at least one tooth of the second cutting blade are configured to by offset from one another by an angle between 10 and 25 degrees.

13. The skin mesher of claim 9 wherein the cutting roller further comprises at least two bearings and the propulsion roller further comprises at least two bearings, wherein the bearings on the cutting roller are located in different positions than the bearings on the propulsion roller.

14. A method of assembling a skin mesher with a cover and a base comprising: opening the cover of the skin mesher, placing a propulsion roller with at least two bearings and a gear within the base by aligning one bearing within a first slot in the base and aligning the second bearing within a second slot in the base, placing a cutting roller with at least two bearings and another gear on top of propulsion roller by aligning one of the bearings with a first circular arc and a second circular arc in the base and aligning the other bearing with a third circular arc and a fourth circular arc in the base and aligning the propulsion roller gear with the cutting roller gear, and closing the cover.
